SEQUENCE LISTING (6 sequences)

SEQ ID NO: 1 - protein, 12 aa, Artificial sequence
  Synthetic peptide
SIPSHSIHSAKA

SEQ ID NO: 2 - protein, 283 aa, Artificial sequence
  BKT-P2-FC polypeptide
MNSFSTSAFGPVAFSLGLLLVLPAAFPAPVPPSIPSHSIHSAKAGGGSKGFEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 233 aa, Artificial sequence
  Human IgG1 Fc (Fc N297A)
FEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 32 aa, Artificial sequence
  IL6 signal peptide
MNSFSTSAFGPVAFSLGLLLVLPAAFPAPVPP

SEQ ID NO: 5 - protein, 6 aa, Artificial sequence
  Hexapeptide spacer sequence
GGGSKG

SEQ ID NO: 6 - DNA, 852 bp, Artificial sequence
  IL6-P2-Fc N297A DNA coding sequence
atgaacagcttcagcaccagcgccttcggccccgtggccttcagcctgggcctgctgctggtgctgcctgccgccttccctgcccccgtgccccccagcatccccagccacagcatccacagcgccaaggccggcggcggcagcaagggcttcgaacccaagagctgtgacaagacccacacctgccccccttgccctgcccctgagctgctgggcggacccagcgtgttcctgttccctcccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacgccagcacctaccgggtggtgtccgtgctgaccgtgctgcaccaggattggctgaacggcaaggaatacaagtgtaaggtgtccaacaaggccctgcctgcccctatcgagaaaaccatcagcaaggccaagggccagcctagggagccccaggtgtacaccctgccccctagcagagatgagctgaccaagaatcaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggataagagcagatggcagcagggcaacgtgttcagctgctccgtgatgcacgaggccctgcacaatcactacacccagaagagcctgagcctgtcccctggcaagtga

(19) United States
(12) Patent Application Publication
(10) Pub. No.: US 2017/0226157 A1
(43) Pub. Date: Aug. 10, 2017

(54) COMPOSITIONS AND METHODS FOR THE TREATMENT OF OCULAR DISEASES

(71) Applicant: Biokine Therapeutics Ltd., Nes Ziona (IL)

(72) Inventor: Amnon PELED, Tel-Aviv (IL)

(21) Appl. No.: 15/326,512

(22) PCT Filed: Dec. 9, 2015
(86) PCT No.: PCT/IL2015/051190

§ 371 Date: Jan. 16, 2017

Related U.S. Application Data
(60) Provisional application No. 62/089,411, filed on Dec. 9, 2014.

(51) Int. Cl. C07K 7/08; C07K 16/00

(52) U.S. Cl. CPC C07K 7/08; C07K 16/00; C07K 2319/30; C07K 2319/02

(57) ABSTRACT

Use of a polypeptide comprising a chemokine binding peptide as set forth in SEQ ID NO: 1 attached to an Fc domain or a fragment thereof is provided. The polypeptide is used in the manufacture of a medicament identified for the treatment of an ocular disease in a subject in need thereof.

### FIELD AND BACKGROUND OF THE INVENTION

The present invention, in some embodiments thereof, relates to compositions and methods for the treatment of ocular diseases.

Age-related macular degeneration (AMD) is the major cause of blindness in the Western World. There are two sub-types of AMD: in atrophic, non-neovascular (aAMD, dry type) a degenerative process involves the retina, retinal pigment epithelium (RPE), and choroid. At its advanced stage aAMD can result in atrophic macular changes and severe visual loss. In neovascular AMD (nvAMD, wet type) the degenerative process is complicated by choroidal neovascularization (CNV), a process which, in the long-term, often leads to substantial visual loss despite application of current anti-vascular endothelial growth factor (VEGF)-based therapies. Additionally, nvAMD is also associated with the development of atrophic macular alterations which result in further visual loss.

Multiple evidences suggest that altered immune response and low-grade chronic inflammation play a critical role in the pathogenesis of both sub types of AMD. Among such evidence are presence of complement component in drusen in the eye, the hallmark of dry AMD, association of single nucleotide polymorphisms (SNPs) in complement factor H (CFH) and in other complement components with the risk for having AMD, and increased expression of markers of inflammation, such as plasma levels of complement components and activation-fragments in AMD patients.

High serum IL-6 and C-reactive protein levels are also associated with early AMD incidence and late AMD.

Chemokines are among the many biological factors that are involved in the inflammatory disease process. Chemokines belong to a group of small, ˜8-14 kDa, mostly basic, heparin-binding proteins that are related both in their primary structure and the presence of 4 conserved cysteine residues.

The chemokines are chemotactic cytokines that have been shown to be selective chemoattractants for leukocyte sub-populations in vitro, and to elicit the accumulation of inflammatory cells in vivo. In addition to chemotaxis, chemokines mediate leukocyte de-granulation, up-regulation of adhesion receptors, and suppression of human immunodeficiency virus replication.

Chemokines play an essential role in the recruitment and activation of cells from the immune system. They also have a wide range of effects in many different cell types beyond the immune system, including for example, in various cells of the central nervous system. Particular chemokines may have multiple effects on tumors, including angiogenesis, promotion of growth and metastasis, and suppression of the immune response to cancer, while other chemokines inhibit tumor-mediated angiogenesis and promote anti-tumor immune responses.

Chemokine receptors have received increasing attention due to their critical role in the progression of inflammation and associated conditions such as asthma, atherosclerosis, graft rejection, AIDS and autoimmune conditions (e.g., multiple sclerosis, arthritis, myasthenia gravis, lupus).

International Patent Publication WO2010/146584 discloses polypeptides comprising a chemokine-binding peptide and an Fc fragment. The polypeptides are capable of binding to certain chemokines so as to modulate their activity.

### SUMMARY OF THE INVENTION

According to an aspect of some embodiments of the present invention there is provided use of a polypeptide comprising a chemokine binding peptide as set forth in SEQ ID NO: 1 attached to an Fc domain or a fragment thereof, in the manufacture of a medicament identified for the treatment of an ocular disease in a subject in need thereof.

According to an aspect of some embodiments of the present invention there is provided a method of treating an ocular disease in a subject in need thereof, the method comprising administering to the subject a therapeutically effective amount of a polypeptide comprising a chemokine binding peptide as set forth in SEQ ID NO: 1 attached to an Fc domain or a fragment thereof, thereby treating the ocular disease in the subject.

According to an aspect of some embodiments of the present invention there is provided a pharmaceutical composition comprising as an active ingredient a polypeptide comprising a chemokine binding peptide as set forth in SEQ ID NO: 1 attached to an Fc domain or a fragment thereof and a pharmaceutically acceptable carrier or diluents formulated for ocular administration.

According to some embodiments of the invention, the ocular disease is selected from the group consisting of age-related macular degeneration (AMD), diabetic retinopathy, retinal vein occlusion, retinal degeneration, retinitis pigmentosa and uveitis.

According to some embodiments of the invention, the ocular disease is age-related macular degeneration (AMD).

According to some embodiments of the invention, the age-related macular degeneration (AMD) is atrophic, non-neovascular (aAMD).

According to some embodiments of the invention, the age-related macular degeneration (AMD) is neovascular.

According to some embodiments of the invention, the administering comprises ocular administration.

According to some embodiments of the invention, the ocular administration comprises a topical administration.

According to some embodiments of the invention, the ocular administration comprises a local ocular administration.

According to some embodiments of the invention, the local ocular administration is selected from the group consisting of subconjunctival, intravitreal, retrobulbar and intracameral administration.

According to some embodiments of the invention, the ocular administration comprises a systemic administration.

According to some embodiments of the invention, the chemokine binding peptide is attached to the Fc domain via a linker.

According to some embodiments of the invention, the linker is an amino acid linker.

According to some embodiments of the invention, the Fc domain is selected from the group consisting of an IgG Fc domain, an IgA Fc domain, an IgD Fc domain, an IgE Fc domain and an IgM Fc domain.

According to some embodiments of the invention, the chemokine-binding peptide is attached to an N terminus of the Fc domain or fragment thereof.

According to some embodiments of the invention, the chemokine-binding peptide is attached to a C terminus of the Fc domain or fragment thereof.

According to some embodiments of the invention, the linker is composed of 4 to 10 amino acids.

According to some embodiments of the invention, the linker is a hexapeptide.

According to some embodiments of the invention, the linker is a peptide set forth in SEQ ID NO: 5.

According to some embodiments of the invention, the chemokine binding peptide is attached to a signal peptide at the N-terminus of the chemokine binding peptide.

According to some embodiments of the invention, the polypeptide is as set forth in SEQ ID NO: 2.

According to some embodiments of the invention, the chemokine is MIP3a.

According to some embodiments of the invention, the chemokine is selected from the group consisting of I-TAC, MIG, RANTES, IP-10, MIP3a, MCP-1, TARK, Eotaxin and SLC.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B are graphs showing that BKT130 (SEQ ID NO: 2) binds chemokines which are relevant to the pathogenesis of AMD and inhibits migration of cells induced by these chemokines.

FIGS. 2A-B show that treatment with BKT130 yields CNV suppression in rats. Daily subcutaneous injections of 1 mg of BKT130 were performed for 10 days following the laser injury. Fluorescein angiogram documented CNV formation (+; lowers panels). RPE-choroid flat mounts stained with isolectin were performed (top panels) and CNV size was measured by a masked observer (FIG. 2A). Analysis demonstrated favorable treatment effect with approximately 30% reduction in mean CNV size (FIG. 2B).

FIGS. 3A-B show subretinal macrophages following anti-chemokine polypharmacology, i.e., BKT130 treatment. RPE-choroid flat mounts were stained for the CD11b macrophage marker (FIG. 3A, pink stain). The analysis showed a reduction of subretinal macrophages (FIG. 3B).

### DESCRIPTION OF SPECIFIC EMBODIMENTS OF THE INVENTION

The present invention, in some embodiments thereof, relates to compositions and methods for the treatment of ocular diseases.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Whilst reducing the present invention to practice, the present inventor has realized that the chemokine binding polypeptide, also termed as BKT130 (set forth in SEQ ID NO: 2) can be used for the treatment of eye diseases. It is suggested that the polypeptide causes choroidal neovascularization (CNV) growth suppression and reduces subretinal macrophage accumulation. Thus the present teachings provide for a new use for the polypeptides described herein.

Thus according to an aspect of the invention there is provided use of a polypeptide comprising a chemokine binding peptide as set forth in SEQ ID NO: 1 attached to an Fc domain or a fragment thereof, in the manufacture of a medicament identified for the treatment of an ocular disease in a subject in need thereof.

Thus according to an aspect of the invention there is provided a method of treating an ocular disease in a subject in need thereof, the method comprising administering to the subject a therapeutically effective amount of a polypeptide comprising a chemokine binding peptide as set forth in SEQ ID NO: 1 attached to an Fc domain or a fragment thereof, thereby treating the ocular disease in the subject.

As used herein a “chemokine” refers to small cytokines, or signaling proteins secreted by cells, which are able to induce directed chemotaxis in nearby responsive cells. The chemokines are classified according to shared structural characteristics such as small size (they are all approximately 8-10 kilodaltons in size), and the presence of four cysteine residues in conserved locations that are key to forming their 3-dimensional shape. Also included in the family of chemokines are SIS family of cytokines, SIG family of cytokines, SCY family of cytokines, Platelet factor-4 superfamily or intercrines. Chemokines according to the present teachings can be pro-inflammatory or homeostatic chemokines. The term chemokines refers to human chemokines however vertebrates, viruses or bacteria chemokines are also contemplated herein. The term is meant to encompass, chemokines of CXC, CC, CX3C and XC families, which exert their biological effects by interacting with G protein-linked chemokine receptors. Specific examples of chemokines are provided hereinbelow.

According to a specific embodiment the chemokine is selected from the group consisting of I-TAC, MIG, RANTES, IP-10, MIP3a, MCP-1, TARK, EOTAXIN, and SLC.

As used herein, the term “chemokine-binding peptide” refers to any peptide (e.g., a peptide of up to 20 amino acid residues) characterized by an ability to bind to at least one chemokine in solution. Exemplary chemokines include, but are not limited to I-TAC (Interferon-inducible T-cell alpha chemoattractant), IP-10 (10 kDa Interferon-γ-induced protein), MIG (Monokine induced by gamma-interferon), MCP-1 (Monocyte chemotactic protein-1), eotaxin, MIP3a, SLC and RANTES (Regulated upon activation, normal T-cell expressed, and secreted).

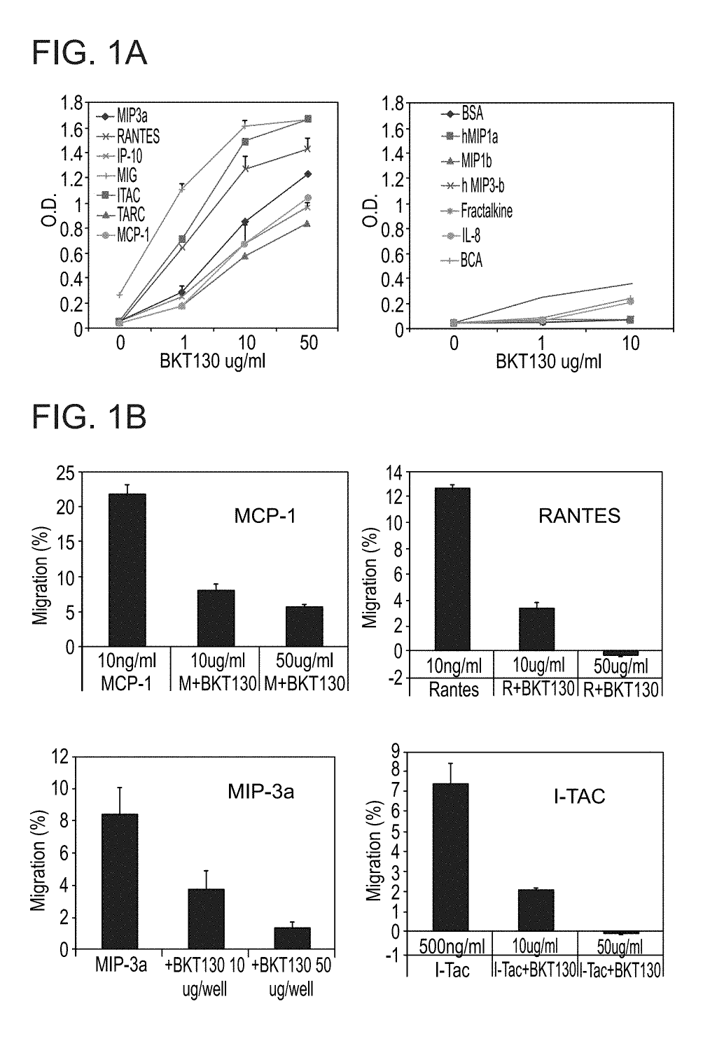

Binding to a chemokine may be determined according to any suitable technique (e.g., ELISA) known in the art. See FIGS. 1A-B for chemokine binding assays. Optionally, the binding is such that a dissociation constant (Ka) for binding of the peptide to a chemokine is less than 10⁻⁴ M, optionally less than 10⁻⁵ M, and optionally less than 10⁻⁶ M. Suitable techniques for determining a dissociation constant will be known to the skilled artisan.

Chemokine-binding peptides which may be used according to embodiments of the invention are described, for example, in International Patent Application PCT/IL03/00155 (published as WO 03/072599) and U.S. Pat. No. 7,488,717.

In some embodiments, the chemokine-binding peptide is from 5 to 50 amino acids in length, optionally from 5 to 40 amino acids in length, optionally from 5 to 30 amino acids in length, optionally from 7 to 20 amino acids in length, optionally from 9 to 20 amino acids in length, and optionally from 10 to 20 amino acids in length. According to exemplary embodiments, the chemokine binding peptide is about 12 amino acids in length.

In some embodiments, the chemokine-binding peptide is characterized by the presence of at least 2 histidine residues and an overall positive charge (e.g., positively charged amino acids outnumber negatively charged amino acids), wherein the peptide is composed primarily of the amino acids selected from the group consisting of H, S, A, L, I, K, R, T and P (e.g., at least 40%, and optionally at least 50% of the peptide consists of the aforementioned amino acids).

According to a specific embodiment the chemokine-binding peptide is SIPSHSIHSAKA (SEQ ID NO: 1), referred to herein as “BKT-P2”, is an exemplary chemokine-binding peptide, which is the chemokine-binding peptide of BKT130 (SEQ ID NO: 2).

In some embodiments, the chemokine-binding peptide is characterized by the presence of at least two adjacent histidine residues and an overall positive charge (e.g., positively charged amino acids outnumber negatively charged amino acids), wherein the peptide is composed primarily of the amino acids selected from the group consisting of H, P, T, L, R, W and F (e.g., at least 40%, and optionally at least 50% of the peptide consists of the aforementioned amino acids).

Optionally, the chemokine binding peptide has an amino acid sequence showing at least 70%, at least 80% at least 90% sequence homology to an amino acid sequence of SEQ ID NO: 1). Optionally, the sequence homology is at least 95%. Optionally, the sequence homology is 100%.

Homology may be determined using BlastP software of the National Center of Biotechnology Information (NCBI) using default parameters. Homology may also refer to a deletion, insertion, or substitution variant, including an amino acid substitution thereof and biologically active polypeptide fragments thereof.

As used herein, an “Fc domain” refers to a region of a heavy chain of an antibody, typically comprising at least 2 constant domains (e.g., CH2 and CH3 domains, as these terms are defined in the art) of the heavy chain. The Fc domain may be obtained, for example, in the form of a dimer, by digestion of an antibody by papain. A dimer of Fc domain polypeptides, connected by disulfide bonds, forms the “tail” region of an antibody. As is known in the art, Fc domains of some classes of antibodies may be in the form of multimers. Thus, the Fc domain is optionally monomeric, optionally dimeric and optionally multimeric. Optionally, the polypeptide described herein is in the form of a dimer, the polypeptide comprising an Fc dimer, or in the form of a multimer, the polypeptide comprising an Fc multimer.

The Fc domain may encompasses modified forms of a native Fc domain (i.e., a domain which occurs naturally in an antibody), for example, polypeptides having at least 90% homology, optionally at least 95% homology, and optionally at least 98% homology, to a native Fc domain. Modified Fc domains are described, for example, in International Patent Applications WO 97/34631 and WO 96/32478.

Optionally, a native Fc is modified so as to remove sites which provide structural features or biological activity that are not required for embodiments of the present invention. Examples of such sites include residues that affect or are involved in disulfide bond formation, incompatibility with a selected host cell, N-terminal heterogeneity upon expression in a selected host cell, glycosylation, interaction with complement, binding to an Fc receptor (other than a neonatal Fc receptor), and/or antibody-dependent cellular cytotoxicity.

The polypeptide according to embodiments of the present invention may also comprise a fragment of an Fc domain. Optionally, the fragment comprises at least 20%, optionally at least 50%, and optionally at least 80% of an Fc domain, as defined hereinabove.

The Fc domain or fragment thereof optionally includes a binding site for a neonatal Fc receptor (FcRn).

As exemplified in the Examples section below, polypeptides according to embodiments of the present invention exhibited a relatively long lifetime in the blood circulation, with significant levels of polypeptide remaining in the blood at least 11 days after administration.

According to one embodiment, attachment of an Fc domain or a fragment thereof to the chemokine-binding peptide results in a polypeptide having a longer half-life in vivo than the chemokine-binding peptide per se. This may be due to the long serum half-life of the Fc domain (which may be due to salvage of the Fc via binding to FcRn) and/or due to the greater size of the polypeptide in comparison to the chemokine-binding peptide, which reduces clearance from the bloodstream by glomerular filtration. According to another embodiment, the resulting polypeptides have reduced immunogenicity as compared to the chemokine-binding peptide per se.

According to optional embodiments, the Fc domain or fragment thereof is a human Fc domain (e.g., derived from a human antibody) or fragment thereof.

According to exemplary embodiments, the Fc domain (or fragment thereof) is an IgG (e.g., IgG1) Fc domain (or fragment thereof). Optionally, the Fc domain or fragment is non-glycosylated.

According to exemplary embodiments, the Fc fragment has SEQ ID NO: 3, which corresponds to a human IgG1 Fc fragment with an N297A mutation.

Optionally, the N-terminus of the Fc domain (or fragment thereof) is attached, (directly or via a linker), to a chemokine-binding peptide. The N-terminus is optionally attached to more than one chemokine-binding peptide, for example, by being attached to a sequence which comprises more than one chemokine-binding peptide.

Alternatively or additionally, the C-terminus of the Fc domain (or fragment thereof) is attached, directly or via a linker (e.g., an amino acid linker), to a chemokine-binding peptide. The C-terminus is optionally attached to more than one chemokine-binding peptide.

In addition to the chemokine-binding peptide and the Fc domain or fragment described herein, the polypeptide described herein may further comprise one or more additional peptide sequences. Such sequences may optionally be selected so as to provide a desirable biological activity or structural feature, for example, an activity or feature which improves a therapeutic efficacy of the polypeptide or which facilitates production of the polypeptide.

Thus, for example, the polypeptide optionally comprises at least one signal peptide selected to be capable of directing transport of the polypeptide.

Optionally, the signal peptide is selected to be capable of promoting secretion of the polypeptide from a cell (e.g., a mammalian cell) expressing the polypeptide.

Optionally, a vacuolar signal sequence (for a polypeptide produced by plant transfection) is included.

In exemplary embodiments, the polypeptide comprises an IL-6 (interleukin-6) signal peptide (e.g., SEQ ID NO: 4) or a signal peptide that has an amino acid sequence showing at least 90% homology (optionally at least 95% homology) to an IL-6 signal peptide.

The above mentioned additional peptide sequence (e.g., signal peptide) may be present at the N-terminus of the polypeptide, the C-terminus of the polypeptide or in the middle of the polypeptide (e.g., between an Fc domain or fragment and a chemokine-binding peptide, or between two chemokine-binding peptides).

Thus, the Fc domain or fragment, the chemokine-binding peptide(s) and any additional peptide(s) (if present) described herein may be attached to one another within the polypeptide in any order.

The Fc domain or fragment, the chemokine-binding peptide(s) and any additional peptide(s) (if present) are independently attached to one another directly or via a linker (e.g., an amino acid linker).

However, the activity of some signal peptides depends on location at a particular location (e.g., N-terminus, C-terminus) within a polypeptide. Hence, according to some embodiments, a signal peptide is located at a particular location in the polypeptide.

According to exemplary embodiments, the signal peptide (e.g., an IL-6 signal peptide) is at the N-terminus of the polypeptide. Examples of polypeptides having a signal peptide at the N-terminus include polypeptides of the following formulas:

W-X-Z, W-X-Y-Z, W-Y-X-Z, W-Y-X-Y-Z, W-Z-X, W-Z-Y-X, W-Y-Z-X and W-Y-Z-Y-X,

wherein W is a signal peptide, X is a chemokine-binding peptide, Y is a linker (e.g., amino acid linker) and Z is the Fc domain or fragment. According to exemplary embodiments, the polypeptide has the above formula W-X-Y-Z.

The linker (also referred to as a “spacer”) is optionally an amino acid linker comprising an amino acid, a dipeptide, a tripeptide, or 4 or more amino acids. Such linkers are advantageous in that they allow the polypeptide to comprise a single contiguous polypeptide chain, which can be readily produced as a fusion protein. The linker is typically selected such that it does not interfere with binding of the chemokine binding peptide to its target chemokine. Optionally, the linker is a peptide composed of 4 to 10 amino acids (e.g., a hexapeptide). According to exemplary embodiments, the linker is a peptide set forth in SEQ ID NO: 5. In embodiments comprising more than one linker, the linkers may be the same or different from one another.

According to preferred embodiments of the invention, the polypeptide is capable of binding to at least one chemokine (e.g., via interaction of the chemokine-binding peptide in the polypeptide with the chemokine). Binding of a polypeptide to a chemokine will in many cases affect the ability of a chemokine (e.g., I-TAC, IP-10, MIG, MCP-1, eotaxin, MIP3a, TARK, SLC and/or RANTES) to bind to a chemokine receptor.

Hence, in some embodiments, the polypeptide is characterized by an ability to inhibit binding of at least one chemokine to a chemokine receptor. Optionally, the inhibition of binding is such that the dissociation constant (for binding of the chemokine to the receptor) in the presence of the polypeptide is at least 10%, and optionally at least 100%, higher than the dissociation constant in the absence of the polypeptide.

In some embodiments, the polypeptide is characterized by an ability to enhance binding of at least one chemokine to a chemokine receptor. Optionally, the enhancement of binding is such that the dissociation constant (for binding of the chemokine to the receptor) in the presence of the polypeptide is at least 10%, and optionally at least 50%, lower than the dissociation constant in the absence of the polypeptide.

It is to be appreciated that a polypeptide according to embodiments of the present invention may optionally inhibit binding of at least one chemokine to a chemokine receptor and also enhance binding of at least one other chemokine to a chemokine receptor.

According to a specific embodiment, the polypeptide binds the chemokine as described herein, and at least one of inhibits chemokine-induced cell migration, causes CNV growth suppression and reduces subretinal macrophage accumulation.

Accordingly, the polypeptide is as set forth in SEQ ID NO: 2 (or a polypeptide as described herein as an Fc fusion comprising SEQ ID NO: 1, 3, 4 and/or 5 e.g., 1).

According to a specific embodiment, the chemokine is selected from the group consisting of I-TAC, MIG, RANTES, IP-10, MIP3a, MCP-1, TARK, Eotaxin and SLC.

According to a specific embodiment, the chemokine is selected from the group consisting of MCP-1, RANTES, MIP-3a and I-TAC.

According to a specific embodiment, the polypeptide inhibits migration induced by a chemokine selected from the group consisting of MCP-1, RANTES, MIP-3a and I-TAC.

The term “polypeptide” as used herein encompasses native polypeptides (e.g., degradation products, synthetically synthesized polypeptides or recombinant polypeptides) and peptidomimetics (typically synthetically synthesized), as well as peptoids and semipeptoids which are polypeptide analogs, which may have, for example, modifications rendering the polypeptides more stable while in a body or more capable of penetrating into cells. Such modifications include, but are not limited to N′ terminus modification, C′ terminus modification, polypeptide bond modification, including, but not limited to, CH2-NH, CH2-S, CH2-S═O, O═C—NH, CH2-O, CH2-CH2, S═C—NH, CH═CH or CF═CH, backbone modifications, and residue modifications. Methods for preparing peptidomimetic compounds are well known in the art and are specified, for example, in Quantitative Drug Design, C. A. Ramsden Gd., Chapter 17.2, F. Choplin Pergamon Press (1992).

Polypeptide bonds (—CO—NH—) within the polypeptide may be substituted, for example, by N-methylated bonds (—N(CH3)-CO—), ester bonds (—C(R)H—C—O—O—C(R)—N—), ketomethylene bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), polypeptide derivatives (—N(R)—CH2-CO—), wherein R is the “normal” side chain, naturally presented on the carbon atom.

These modifications can occur at any of the bonds along the polypeptide chain and even at several (2-3) at the same time.

Natural aromatic amino acids, Trp, Tyr and Phe, may be substituted for synthetic non-natural acids such as phenylglycine, 1,2,3,4-tetrahydroisoquinoline-3-carboxylic acid (TIC), naphthylalanine (Nal), ring-methylated derivatives of Phe, halogenated derivatives of Phe or o-methyl-Tyr.

The amino acid sequences of naturally occurring peptides and polypeptides described herein (e.g., Fc domain sequences, signal peptides) may either be the amino acid sequences of the polypeptides in naturally-occurring proteins or those that comprise either conservative or non-conservative substitutions.

The term “conservative substitution” as used herein, refers to the replacement of an amino acid present in the native sequence in the peptide with a naturally or non-naturally occurring amino acid or a peptidomimetic having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered conservative substitutions.

For producing conservative substitutions by non-naturally occurring amino acids it is also possible to use amino acid analogs (synthetic amino acids) well known in the art. A peptidomimetic of the naturally occurring amino acids is well documented in the literature known to the skilled practitioner.

When effecting conservative substitutions the substituting amino acid should have the same or a similar functional group in the side chain as the original amino acid.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cyclohexylmethyl glycine for alanine, isoleucine for glycine, or —NH—CH[(—CH₂)₅COOH]—CO— for aspartic acid. Those non-conservative substitutions which fall within the scope of the present invention are those which still constitute a peptide or polypeptide having an activity of the native peptide (e.g., Fc domain sequence, signal peptide).

As used herein in the specification and in the claims section below, the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Recombinant techniques are preferably used to generate the polypeptides of the present invention since these techniques are better suited for generation of relatively long polypeptides (e.g., longer than 20 amino acids) and large amounts thereof. Such recombinant techniques are described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680, Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

To produce a polypeptide of the present invention using recombinant technology, a polynucleotide encoding a polypeptide of the present invention is ligated into a nucleic acid expression vector, which comprises the polynucleotide sequence under the transcriptional control of a cis-regulatory sequence (e.g., promoter sequence) suitable for directing constitutive, tissue specific or inducible transcription of the polypeptides of the present invention in the host cells.

An exemplary polynucleotide sequence which can be used to express a polypeptide according to some embodiments of the present invention is set forth in SEQ ID NO: 6 or a functional homolog of same e.g., at least 80%, 85%, 90%, 95% identical to SEQ ID NO: 6 (e.g., codon optimized).

The phrase “an isolated polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

As used herein, the phrase “complementary polynucleotide sequence” refers to a sequence which results from reverse transcription of messenger RNA using a reverse transcriptase or any other RNA-dependent DNA polymerase. Such a sequence can be subsequently amplified in vivo or in vitro using a DNA-dependent DNA polymerase.

As used herein, the phrase “genomic polynucleotide sequence” refers to a sequence derived (isolated) from a chromosome and thus represents a contiguous portion of a chromosome.

As used herein, the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis-acting expression regulatory elements.

As mentioned, the polynucleotides of embodiments of the present invention may further comprise a signal sequence encoding a signal peptide (as discussed hereinabove) for the secretion of the polypeptide.

Following expression and secretion, the signal peptides are optionally removed from the precursor proteins resulting in the mature polypeptide.

Polynucleotides of the present invention may be prepared using PCR techniques as described, for example, in Example 1 herein below, or any other method or procedure known in the art for ligation of two different DNA sequences. See, for example, “Current Protocols in Molecular Biology”, eds. Ausubel et al., John Wiley & Sons, 1992.

As mentioned hereinabove, polynucleotide sequences of the present invention are typically inserted into expression vectors (i.e., a nucleic acid construct) to enable expression of the recombinant polypeptide. The expression vector of embodiments of the present invention includes additional sequences which render this vector suitable for replication and integration in prokaryotes, eukaryotes, or preferably both (e.g., shuttle vectors). Typical cloning vectors contain transcription and translation initiation sequences (e.g., promoters, enhances) and transcription and translation terminators (e.g., polyadenylation signals).

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000-fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of the present invention can further include additional polynucleotide sequences that allow, for example, the translation of several proteins from a single mRNA such as an internal ribosome entry site (IRES) and sequences for genomic integration of the promoter-chimeric polypeptide.

Examples of mammalian expression vectors include, but are not limited to, pcDNA3, pcDNA3.1(+/−), pGL3, pZeoSV2(+/−), pSeeTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pIRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p205. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed.

Recombinant viral vectors may be useful for expression of the polypeptides of embodiments of the present invention since they offer advantages such as lateral infection. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type infection in which the infectious agent spreads only through daughter progeny. Viral vectors can also be produced that are unable to spread laterally. This characteristic can be useful if the desired purpose is to introduce a specified gene into only a localized number of targeted cells.

A variety of prokaryotic or eukaryotic cells can be used as host-expression systems to express the polypeptides of the present invention. These include, but are not limited to, microorganisms, such as bacteria (for example, E. coli including but not limited to E. coli strains BL21 (DE3) plysS, BL21;(DE3)RP and BL21* and B. subtilis) transformed with a recombinant bacteriophage DNA, plasmid DNA or cosmid DNA expression vector containing the polypeptide coding sequence; yeast transformed with recombinant yeast expression vectors containing the polypeptide coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the polypeptide coding sequence.

Various methods can be used to introduce the expression vector of the present invention into the cells of the host expression system. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Thus, the present invention encompasses nucleic acid sequences described hereinabove; fragments thereof, sequences hybridizable therewith, sequences homologous thereto, sequences orthologous thereto, sequences encoding similar polypeptides with different codon usage, altered sequences characterized by mutations, such as deletion, insertion or substitution of one or more nucleotides, either naturally occurring or man induced, either randomly or in a targeted fashion.

The polypeptides of embodiments of the present can be conveniently purified by affinity chromatography. The suitability of protein A as an affinity ligand depends on the species and isotype of the immunoglobulin Fc domain that is used in the chimera. Protein A can be used to purify chimeric molecules that are based on human γ1, γ2, or γ4 heavy chains [Lindmark et al., J. Immunol. Meth., 62:1-13 (1983)]. Protein G is preferably used for all mouse isotypes and for human γ3 [Guss et al., EMBO J., 5:1567-1575 (1986)]. The solid support to which the affinity ligand is attached is most often agarose, but other solid supports are also available. Mechanically stable solid supports such as controlled pore glass or poly(styrenedivinyl)benzene allow for faster flow rates and shorter processing times than can be achieved with agarose. The conditions for binding the chimeric molecules to the protein A or G affinity column are dictated entirely by the characteristics of the Fc domain; that is, its species and isotype. Generally, when the proper ligand is chosen, efficient binding occurs directly from unconditioned culture fluid. Bound polypeptides of this aspect of the present invention can be efficiently eluted either at acidic pH (at or above 3.0), or in a neutral pH buffer containing a mildly chaotropic salt. This affinity chromatography step can result in polypeptide preparation that is >95% pure.

Other methods known in the art can be used in place of, or in addition to, affinity chromatography on protein A or G to purify the polypeptides of embodiments of the present invention. For example, it is anticipated that the polypeptides may behave similarly to antibodies in thiophilic gel chromatography [Hutchens et al., Anal. Biochem., 159:217-226 (1986)] and immobilized metal chelate chromatography [Al-Mashikhi et al., J. Dairy Sci., 71:1756-1763 (1988)].

To facilitate recovery, the expressed coding sequence can be engineered to encode the polypeptide of the present invention and fused cleavable moiety e.g. histidine. Such a fusion protein can be designed so that the polypeptide can be readily isolated by affinity chromatography; e.g., by immobilization on a column specific for the cleavable moiety.

Where a cleavage site is engineered between the polypeptide and the cleavable moiety, the polypeptide can be released from the chromatographic column by treatment with an appropriate enzyme or agent that specifically cleaves the fusion protein at this site [e.g., see Booth et al., Immunol. Lett. 19:65-70 (1988); and Gardella et al., J. Biol. Chem. 265:15854-15859 (1990)].

The polypeptide of the present invention is preferably retrieved in a “substantially pure” form.

As used herein, the phrase “substantially pure” refers to a purity that allows for the effective use of the polypeptide in the applications described herein.

In addition to being synthesizable in host cells, the polypeptide of the present invention can also be synthesized using in vitro expression systems. These methods are well known in the art and the components of the system are commercially available.

The polypeptide may be used as is, or further modified.

For example, the polypeptide may be modified by attaching a water-soluble polymer (e.g., polyethylene glycol) to the polypeptide. Such an attachment of a water-soluble polymer may improve a biological activity of the polypeptide, for example, by increasing a solubility of the polypeptide, reducing a toxicity of the polypeptide, extending the circulation half-life of the polypeptide (e.g., by reducing glomerular filtration), and/or protecting the polypeptide from proteolytic degradation.

Hence, according to optional embodiments of the present invention, the polypeptide described herein is attached to a water-soluble polymer. Polyethylene glycol is a suitable water-soluble polymer. Additional water-soluble polymers suitable (e.g., non-toxic) for attaching to a polypeptide intended for pharmaceutical administration will be apparent to one of skill in the art.

The water-soluble polymer may be attached to any part of the polypeptide (directly or via a linker). Optionally, the water-soluble polymer is attached to a part of the polypeptide other than the chemokine-binding peptide, so as not to interfere with the chemokine-binding activity thereof.

Methods and uses comprising the administration of these polypeptides (e.g., BKT130) find application in treating patients with all types of ocular disorders in which chemokines (e.g., MIP3a, IP10, MIG, ITAC, TARK, RANTES, MCP-1, SLC and Eotaxin) play a role in onset or progression of the disease.

According to some embodiments, these diseases or conditions are characterized by changes in the ocular vasculature whether progressive or non-progressive, whether a result of an acute disease or condition, or a chronic disease or condition.

According to a specific embodiment, the ocular disease is selected from the group consisting of diabetic macular edema, age-related macular degeneration (wet form or dry form), choroidal neovascularization, diabetic retinopathy, ocular ischemia, uveitis, retinal vein occlusion (central or branch), retinal degeneration, ocular trauma, surgery induced edema, surgery induced neovascularization, cystoid macular edema, ocular ischemia and uveitis.

According to a specific embodiment, the ocular disease is selected from the group consisting of age-related macular degeneration (AMD), diabetic retinopathy, retinal vein occlusion, retinal degeneration, retinitis pigmentosa and uveitis.

According to a specific embodiment, the ocular disease is age-related macular degeneration (AMD).

According to a specific embodiment, the age-related macular degeneration (AMD) is atrophic, non-neovascular (aAMD).

According to a specific embodiment, the age-related macular degeneration (AMD) is neovascular.

Also contemplated are all forms of glaucoma, and pigmentary dispersion syndrome, pseudo-exfoliation syndrome, secondary angle-closure glaucoma, neovascular glaucoma, degenerative optic neuropathy and other eye pathologies characterized by a progressive loss of vision leading finally to blindness known to those skilled in the art.

Such conditions have in common a progressive decline of eyesight related to a degenerative process of retina or optic nerve. Treatment is possible at all stages of disease progression including very early stages as a prophylactic. The clinical effects may be two-fold, first an acute improvement in eyesight in those patients already suffering from a glaucomatous degeneration, and second a slowing-down or stopping of the progressive worsening of the eyesight. Even patients who experience blindness in one or two eyes related to glaucoma may regain eyesight to some extent.

As used herein the term “a subject” or “a subject in need thereof” relates to a mammal e.g., human, of any gender or age who is diagnosed with an ocular disease or is at risk of developing such, for instance has been exposed to an agent that is associated with the onset or progression of an ocular disease.

Subjects who may be treated include primate (including humans), canine, feline, ungulate (e.g., equine, bovine, swine (e.g., pig)), avian, and other subjects. Humans and non-human animals having commercial importance (e.g., livestock and domesticated animals) are of particular interest. Exemplary mammals which may be treated include, canines; felines; equines; bovines; ovines; rodentia, etc. and primates, particularly humans. Non-human animal models, particularly mammals, e.g. primate, murine, lagomorpha, etc. may be used for experimental investigations.

According to one embodiment, the subject who is treated is aged 55 or older.

Any mode of topical, local ocular and systemic administration of the polypeptide is contemplated herein.

The polypeptide can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the polypeptide accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Pharmaceutical formulations suitable for ocular administration of the polypeptide of the invention are well known in the art. Some exemplary ophthalmic viscosity enhancers that can be used in the present formulation include: carboxymethyl cellulose sodium; methylcellulose; hydroxypropyl cellulose; hydroxypropylmethyl cellulose; hydroxyethyl cellulose; polyethylene glycol 300; polyethylene glycol 400; polyvinyl alcohol; and providone.

Some natural products, such as veegum, alginates, xanthan gum, gelatin, acacia and tragacanth, may also be used to increase the viscosity of ophthalmic solutions.

A tonicity is important because hypotonic eye drops cause an edema of the cornea, and hypertonic eye drops cause deformation of the cornea. The ideal tonicity is approximately 300 mOsM. The tonicity can be achieved by methods described in Remington: The Science and Practice of Pharmacy, known to those versed in the art.

Suitable ophthalmic carriers are known to those skilled in the art and all such conventional carriers may be employed in the present invention. Exemplary compounds incorporated to facilitate and expedite transdermal delivery of topical compositions into ocular or adnexal tissues include, but are not limited to, alcohol (ethanol, propanol, and nonanol), fatty alcohol (lauryl alcohol), fatty acid (valeric acid, caproic acid and capric acid), fatty acid ester (isopropyl myristate and isopropyl n-hexanoate), alkyl ester (ethyl acetate and butyl acetate), polyol (propylene glycol, propanedione and hexanetriol), sulfoxide (dimethylsulfoxide and decylmethylsulfoxide), amide (urea, dimethylacetamide and pyrrolidone derivatives), surfactant (sodium lauryl sulfate, cetyltrimethylammonium bromide, polaxamers, spans, tweens, bile salts and lecithin), terpene (d-limonene, alphaterpeneol, 1,8-cineole and menthone), and alkanone (N-heptane and N-nonane). Moreover, topically-administered compositions comprise surface adhesion molecule modulating agents including, but not limited to, a cadherin antagonist, a selectin antagonist, and an integrin antagonist. Thus, a particular carrier may take the form of a sterile, ophthalmic ointment, cream, gel, solution, or dispersion. Also including as suitable ophthalmic carriers are slow release polymers, e.g., “Ocusert” polymers, “Hydron” polymers, etc.

Stabilizers may also be used such as, for example, chelating agents, e.g., EDTA. Antioxidants may also be used, e.g., sodium bisulfite, sodium thiosulfite, 8-hydroxy quinoline or ascorbic acid. Sterility typically will be maintained by conventional ophthalmic preservatives, e.g., chiorbutanol, benzalkonium chloride, cetylpyridium chloride, phenyl mercuric salts, thimerosal, etc., for aqueous formulations, and used in amounts which are nontoxic and which generally vary from about 0.001 to about 0.1% by weight of the aqueous solution. Conventional preservatives for ointments include methyl and propyl parabens. Typical ointment bases include white petrolatum and mineral oil or liquid petrolatum. However, preserved aqueous carriers are preferred. Solutions may be manually delivered to the eye in suitable dosage form, e.g., eye drops, or delivered by suitable microdrop or spray apparatus typically affording a metered dose of medicament. Examples of suitable ophthalmic carriers include sterile, substantially isotonic, aqueous solutions containing minor amounts, i.e., less than about 5% by weight hydroxypropylmethylcellulose, polyvinyl alcohol, carboxymethylcellulose, hydroxyethylcelullose, glycerine and EDTA. The solutions are preferably maintained at substantially neutral pH and isotonic with appropriate amounts of conventional buffers, e.g., phosphate, borate, acetate, tris.

In some embodiments, penetration enhancers are added to the ophthalmologic carrier.

Systemic and local routes of administration useful in the methods of the invention encompass, without limitation, oral gavage; intravenous injection; intraperitoneal injection; intramuscular injection; subcutaneous injection; transdermal diffusion and electrophoresis; topical eye drops, creams and ointments; periocular and intraocular injection including subconjunctival injection; extended release delivery devices including locally implanted extended release devices; and intraocular and periocular implants including bioerodible and reservoir-based implants.

In some embodiments, an ophthalmic composition containing the polypeptide as described herein is administered topically to the eye. The ophthalmic composition can be for example, an ophthalmic solution (ocular drops). In other embodiments, the ophthalmic composition containing the polypeptide as described herein is injected directly into the eye. In a further embodiment, the ophthalmic composition containing the polypeptide as described herein is released from an intraocular or periocular implant such as a bioerodible or reservoir-based implant.

In some embodiments, the ophthalmic composition comprising a polypeptide as described herein is administered locally in an extended release formulation. For example, an ophthalmic composition containing a polypeptide as described herein can be administered via an intraocular or periocular implant, which can be, for example, bioerodible or reservoir-based.

As used herein, the term “implant” refers to any material that does not significantly migrate from the insertion site following implantation. An implant can be biodegradable, non-biodegradable, or composed of both biodegradable and non-biodegradable materials; a non-biodegradable implant can include, if desired, a refillable reservoir. Implants useful in the methods of the invention include, for example, patches, particles, sheets, plaques, microcapsules and the like, and can be of any shape and size compatible with the selected site of insertion, which can be, without limitation, the posterior chamber, anterior chamber, suprachoroid or subconjunctiva. It is understood that an implant useful in the invention generally releases the implanted pharmaceutical composition at an effective dosage to the eye of the subject over an extended period of time. A variety of ocular implants and extended release formulations suitable for ocular release are well known in the art, as described, for example, in U.S. Pat. Nos. 5,869,079 and 5,443,505.

In instances where the polypeptide as described herein is encoded by a nucleic acid molecule, administration of a pharmaceutical composition containing the nucleic acid molecule can be carried out using one of numerous methods well known in the art of gene therapy. Such methods include, but are not limited to, lentiviral transformation, adenoviral transformation, cytomegaloviral transformation, microinjection and electroporation. All routes or administrations/formulations are relevant also for the administration of the nucleic acid molecule.

In some embodiments, the polypeptide as described herein are administered to the subject in a liquid or gel suspension in the form of drops, spray or gel. In yet another embodiment, the polypeptide as described herein is injected directly into the lacrimal tissues or onto the eye surface.

In other embodiments, the polypeptide as described herein is applied to the eye via liposomes. In still other embodiments, the polypeptide as described herein is infused into the tear film via a pump-catheter system. I n still another embodiment, the polypeptide as described herein is contained within a continuous or selective-release device, for example, membranes such as, but not limited to, those employed in the Ocusert™ System (Alza Corp., Palo Alto, Calif.). As an additional embodiment, the polypeptide as described herein is contained within, carried by, or attached to contact lenses which are placed on the eye. In yet another embodiment, the polypeptide as described herein is contained within a swab or sponge which can be applied to the ocular surface. Another embodiment of the present invention involves the active compound contained within a liquid spray which can be applied to the ocular surface.

Pharmaceutical compositions suitable for use in context of some embodiments of the invention include compositions wherein the active ingredients are contained in an amount effective to achieve the intended purpose. More specifically, a therapeutically effective amount means an amount of active ingredients (polypeptide) effective to prevent, alleviate or ameliorate symptoms of a disorder (e.g., ocular disease) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide therapeutic (in the eye tissue) levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more units dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The instant methods optionally comprise administering at least one additional pharmaceutical agent which is known in the art to be effective in the treatment of ocular disorders. These additional agents may be selected from the general class of anti-glaucoma drugs including those mentioned above, antibiotics virostatics, steroids, anti-allergic drugs, artificial tears and other drugs used for local and systemic eye treatment. Representative anti-glaucoma drugs include acetazolamide, diclofenamide, carteolol, timolol, metipranolol, betaxolol, pindolol, levobunolol, brimonidine, clonidine, pilocarpine, carbachol, dipivefrine, apraclonidine, brinzolamide, dorzolaminde, bimatoprost, travaprost, and latanoprost. Representative antibiotics used for eye infections are chlortetracycline, ciprofloxacine, ofloxacine, fusidinic acid, gentamicine, kanamycine, levofloxacine, lomefloxacine, oxytetracycline, natamycine, azidamfenicole, chloramphenicole, tobramycine, erythromycine, and polymyxin-B. Representative virostatics include acaclovir, and trifluridine. Representative steroids include betamethasone, dexamethasone, fluorometholone, hydrocortisone, prednisolone, and rimexolone. Representative antiallergic drugs include cromoglicate, azelastine, lodoxamide, emedastine, nedocromile, levocabastine, olopatadinea and ketotifene. Representative artificial tears include hypromellose, carbomere, hyaluronate, carmellose, hypromellose, povidone, hyetellose, polivinylalcohole, and dexpanthenole. Other representative commonly used eye therapeutics are tetryzoline, troxerutine, tramazoline, naphazoline, xylometazoline, phenylephrine, antazoline.

As used herein the term “about” refers to ±10% .

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., Ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (Eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., Ed. (1994); “Culture of Animal Cells—A Manual of Basic Technique” by Freshney, Wiley-Liss, N. Y. (1994), Third Edition; “Current Protocols in Immunology” Volumes I-III Coligan J. E., Ed. (1994); Stites et al. (Eds.), “Basic and Clinical Immunology” (8th Edition), Appleton & Lange, Norwalk, Conn. (1994); Mishell and Shiigi (Eds.), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., Ed. (1984); “Nucleic Acid Hybridization” Hames, B. D., and Higgins S. J., Eds. (1985); “Transcription and Translation” Hames, B. D., and Higgins S. J., Eds. (1984); “Animal Cell Culture” Freshney, R. I., Ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Experimental Procedures

1.1. ELISA

Enzyme-linked-immunosorbent-assay (ELISA) was performed in order to evaluate the binding of BKT130 to different chemokines.

Plates were pre-coated with different chemokines at concentration of 150 ng/well/100 μl in coating buffer (0.1M Carbonate pH 9.5) and incubated over night at 4° C. Plates were washed once with washing buffer (PBS with 0.05% Tween) and blocked with 200 μl of blocking buffer (4% BSA in PBS). Following 1 hour incubation at RT the plates were washed once with washing buffer and 100 μl of BKT130 (0, 1, 10 and 50 ug/ml) were added into each well. Following 2 hours of incubation at RT plates were washed five times with washing buffer and second antibody (Goat anti-Human IgG HRP conjugate) was added at dilution of 1:1000 for 2 hours at RT. Following 2 hr incubation plates were washed five times with washing buffer and 3,3′-5,5′-tetramethylbenzidine (TMB) substrate was added (100 μl/well). 30 minutes later the reaction was stop with 50 μl of stop solution (1M H₂SO4) and the absorbance was measured by ELISA Reader at wavelength of 450 nm.

In order to check the specific binding of the BKT130 to the chemokines, samples of BKT130 were added also to plates that were pre-coated with 4% BSA.

1.2. Migration

Migration assay was performed using transmigration plates of 6.5 mm/diameter and 5 μm/pore. Cells were re-suspended in RPMI medium contains 1% FCS.

600 μl of RPMI medium were added to the lower chambers of the transwells, supplemented with different chemokines (see table 1). BKT130 was added to the lower chambers at final concentration of 10 and 50 ug/ml. The chemokines were incubated with BKT130 for 30 min before the initiation of the migration assay.

Following 30 min of incubation 2×10⁵ of the appropriate type of cells/well were added to the upper chambers of the transmigration plates in total volume of 100 μl.

Cells migrating within 3 hours to the bottom chamber of the Transwell were counted using FACScalibur.

When Jurkat-CXCR3 cells were used the Transwells were pre-coated with fibronectin (FN) prior to migration assay. FN was diluted to 1 mg/ml with PBS and was added to the upper wells in total volume of 100 μl/well. Plates were incubated for 1 hr in the incubator, at 37° C.

CD4+ T cells were obtained from fresh whole blood using Ficoll-Histopaque 1077. CD4+ T cells were further isolated with enrichment Rosette, according to the manufacturer's instructions.

TABLE 1
Final concentration of the different chemokines in the transmigration
assay and the target cells which were tested for each chemokine.

| Chemokine | Concentration | | Cells |
|---|---|---|---|
| I-TAC | 500 | ng/ml | Jurkat-CXCR3 |
| RANTES | 10 | ng/ml | THP-1 |
| MIP3a | 2 | ug/ml | Human CD4+ T cells |
| MCP-1 | 10 | ng/ml | THP-1 |

1.3. Staining of Macrophages with CD11b

Retinal flat mounts were immunohistochemistry stained for CD11b as follows:

Eyes were enucleate and fixed with 4% paraformaldayde (PFA). Following 1 hr of incubation retinas were gently removed and the flat mounts were separated.

Tissues were washed 3 times with PBS for 10 minutes and then blocked for 4 hr with blocking solution (10% NDS, 3% BSA, 0.1% Triton-x).

Cd11b antibody (ab64347—rat monoclonal—abcam) was added at 1:100 in AB solution (3% NDS, 1% BSA in PBS), Over Night at 4° C., on shaker.

Following incubation tissues were washed 6 times for 20 minutes.

Second antibody (ab150154—donkey anti rat alexa fluor 555-cy3) was added in AB solution for 2 hr. Following incubation tissues were washed 6 times for 20 minutes.

Number of CD11b+cells was counted using confocal microscope.

### Treatment of AMD Using BKT130

Antagonizing chemokine as a therapeutic strategy for the treatment of AMD was done using the rat model of laser induced CNV (Choroidal Neovascularization). BKT130 (SEQ ID NO: 2), a recombinant polypeptide which is a promiscuous chemokine antagonists that binds inflammatory chemokines MCP-1,MIP-3A, IP-10, Mig, I-TAC, TARK, Eotaxin, SLC and RENTES (FIG. 1A) and inhibits the migration of immune cells in response to these chemokines (FIG. 1B) was used as the drug lead. An ELISA assay for the various chemokines was used to test binding. A migration assay was done in transwells using THP1 myelomonoctic cells (for MCP1, RANTES) and purified T cells (for MIP-3A, IP-10, Mig, I-TAC, TARK).

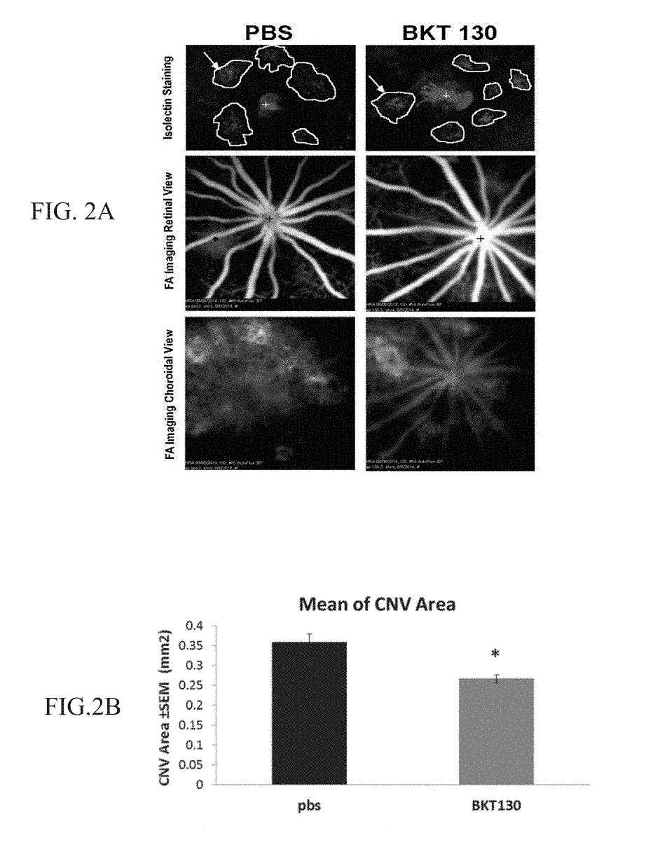

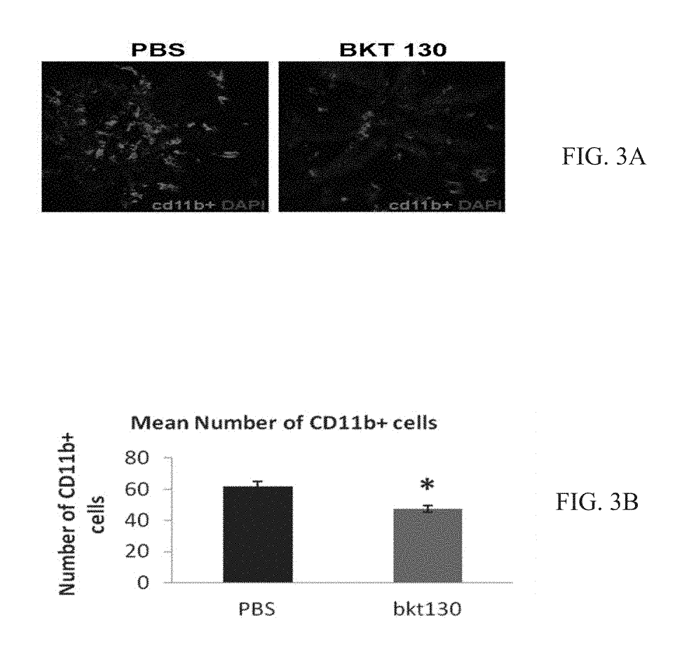

Treatment with BKT130 yielded CNV growth suppression (FIGS. 2A-B) and reduction in sub retinal macrophage accumulation (FIGS. 3A-B).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. (canceled)

2. A method of treating an ocular disease in a subject in need thereof, the method comprising administering to the subject a therapeutically effective amount of a polypeptide comprising a chemokine binding peptide as set forth in SEQ ID NO: 1 attached to an Fc domain or a fragment thereof, thereby treating the ocular disease in the subject.

3. The method of claim 2, wherein said ocular disease is selected from the group consisting of diabetic macular edema, age-related macular degeneration, choroidal neovascularization, diabetic retinopathy, ocular ischemia, uveitis, retinal vein occlusion (central or branch), retinal degeneration, ocular trauma, surgery induced edema, surgery induced neovascularization, cystoid macular edema, ocular ischemia and uveitis.

4. The method of claim 2, wherein said ocular disease is age-related macular degeneration (AMD).

5. The method of claim 3, wherein said age-related macular degeneration (AMD) is atrophic, non-neovascular (aAMD).

6. The method of claim 3, wherein said age-related macular degeneration (AMD) is neovascular.

7. The method of claim 2, wherein said administering comprises ocular administration.

8. The method of claim 7, wherein said ocular administration comprises a topical administration.

9. The method of claim 7, wherein said ocular administration comprises a local ocular administration.

10. The method of claim 9, wherein said local ocular administration is selected from the group consisting of subconjunctival, intravitreal, retrobulbar and intracameral administration.

11. The method of claim 8, wherein said ocular administration comprises a systemic administration.

12. The method of claim 2, wherein said chemokine binding peptide is attached to said Fc domain via a linker.

13. The method of claim 12, wherein said linker is an amino acid linker.

14. The method of claim 2, wherein said Fc domain is selected from the group consisting of an IgG Fc domain, an IgA Fc domain, an IgD Fc domain, an IgE Fc domain and an IgM Fc domain.

15. The method of claim 2, wherein said chemokine-binding peptide is attached to an N terminus of said Fc domain or fragment thereof.

16. The method of claim 13, wherein said linker is composed of 4 to 10 amino acids.

17. The method of claim 16, wherein said linker is a hexapeptide.

18. The method of claim 13, wherein said linker is a peptide set forth in SEQ ID NO: 5.

19. The method of claim 2, wherein said chemokine binding peptide is attached to a signal peptide at the N-terminus of said chemokine binding peptide.

20. The method of claim 2, wherein said polypeptide is as set forth in SEQ ID NO: 2.

21-22. (canceled)

23. The method of claim 2, wherein said polypeptide is as set forth in SEQ ID NO: 2 and said ocular disease is age-related macular degeneration (AMD).
